SEQUENCE LISTING (10 sequences)

SEQ ID NO: 1 - protein, 396 aa, Artemisia vulgaris
MEKHYFVILFTAAFVFVGAAARADIGDELEAAQFNSTRRGLHECAAHNIIDKCWRCKADWEKNRQALAKCAQGFAKGTTGGLGGEIYVVTDCSDDNAANPKPGTLRCGVTQDKPLWIIFKKDMVIKLKHELVINKDKTIDGRGANVEITCGGLTIHNVCNVIIHNIHIHDIKVTEGGIIKATDAKPGHRHKSDGDGICVAGSSKIWIDHCTLSHGPDGLIDVTLGSTAVTISNCKFSHHQKILLLGADNSHVDDKKMHVTVAFNRFAEACDQRMPRCRFGFFQVVNNDYTSWGTYAIGGSANPTILSQGNRFHAPNDPMKKNVLVRADAPHTESMKWNWRSEKDLLENGAIFVASGCDPHLTPEQKSHLIPAEPGSAVLQLTSCAGTLKCVPGKPC

SEQ ID NO: 2 - DNA, 1188 bp, Artemisia vulgaris
atggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttgattccagctgaaccaggatcagcagttcttcaactcaccagttgtgctggcacgctcaaatgcgttcctggaaaaccttgt

SEQ ID NO: 3 - DNA, 1188 bp, Artemisia vulgaris
atggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatcttcaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaacggaaggtggaattattaaggcaacggacgctaaaccagggcatagacataagagcgacggagatggtatttgtgttgctggttcttcgaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtcgcattcaacaggttcgcagaagcatgtgatcaaagaatgccacgatgtcgatttggatttttccaagttgttaacaatgactacaccagctggggaacgtacgccattggtggtagcgccaatcctactatccttagccaaggcaaccgattccatgctcccaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttaattccagctgaaccaggatcagcagttcttcaactcaccagttgtgctggcacgctcaaatgcgttcctggaaaaccttgt

SEQ ID NO: 4 - DNA, 41 bp, Artificial Sequence
  Description of Artificial Sequence Primer
gagagagaccatggctcgggctgacattggtgatgagctcg

SEQ ID NO: 5 - DNA, 42 bp, Artificial Sequence
  Description of Artificial Sequence Primer
gagagagactcgagttaacaaggttttccaggaacgcatttg

SEQ ID NO: 6 - DNA, 1523 bp, Artemisia vulgaris
ggaattcggcacgagaaatctacaaaaattgataaaaataaaaataacaaagcgagtcattggcttgcatacatggctacatgattcgctttagacaacacaataaataatcagcaatatataaagtaccttcggtactttgagatagaaagttgtaaaaaaagataatcatacaatacaatggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttgattccagctgaaccaggatcagcagttcttcaactcaccagttgtgctggcacgctcaaatgcgttcctggaaaaccttgttaatagttatcacccactttttttttttattgttattagcattttttatacttgttaggattgtagtggaatgagacattgatacgtgcattacaagaccattcatcaacatattttgctactatcacatgttcacgttaaaaaaaaaaaaaaaa

SEQ ID NO: 7 - DNA, 1513 bp, Artemisia vulgaris
ggaattcggcacgagaatctacaaaaattgataaaaataaaaataaaaataacaaagcgagtcattggcttgcatacatggctacatgattcgctttagacaacacaataaataatcagcaatatataaagtaccttcggtactttgagatagaaagttgtaaaaaaagataatcatacaatacaatggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttgattccagctgaaccaggatcagcagttcttcaactcaccagttgtgctggcacgctcaaatgcgttcctggaaaaccttgttaatagttatcacccactttttatttttattgttattagcattttttatacttgttaggattgtagtggaatgagacattgatacgtgcattacaagaccattcatcaacatattttgctaaaaaaaaaaaaaaaaaaaa

SEQ ID NO: 8 - DNA, 1520 bp, Artemisia vulgaris
gcggccgctctagaactagtggatcccccgggctgcaggaattcggcacgagaaattgataaaaataaaaataaaaataacaaagcgagtcattggcttgcatacatggctacatgattcgctttagacaacacaataaataatcagcaatatataaagtaccttcggtactttgagatagaaagttgtaaaaaaagataatcatacaatacaatggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatcttcaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaacggaaggtggaattattaaggcaacggacgctaaaccagggcatagacataagagcgacggagatggtatttgtgttgctggttcttcgaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtcgcattcaacaggttcgcagaagcatgtgatcaaagaatgccacgatgtcgatttggatttttccaagttgttaacaatgactacaccagctggggaacgtacgccattggtggtagcgccaatcctactatccttagccaaggcaaccgattccatgctcccaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttaattccagctgaaccaggatcagcagttcttcaactcaccagttgtgctggcacgctcaaatgcgttcctggaaaaccttgttaatagttatcacccactttttatttttattgttattagcattttttatacttgttaggattgtagtggaatgagacattgatacgtgcattacaagaccaaaaaaaaaaaaaaaaaaa

SEQ ID NO: 9 - DNA, 1535 bp, Artemisia vulgaris
ggcggccgctctagaactagtggatcccccgggctgcaggaattcggcacgagtgaaaatctacaaaaattgataaaaataaaaataaaaataacaaagcgagtcattggcttgcatacatggctacatgattcgctttagacaacacaataaataatcagcaatatataaagtaccttcggtactttgagatagaaagttgtaaaaaaagataatcatacaatacaatggaaaaacattattttgttatattgttcaccgcagcgtttgttttcgtgggtgcagctgctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttgattccagctgaaccaggatcagcagttcttcaactcaccagttgtgctggcacgctcaaatgcgttcctggaaaaccttgttaatagttatcacccactttttatttttattgttattagcattttttatacttgttaggattgtagtggaatgagacattgatacgtgcattacaagaccaaaaaaaaaaaaaaaaaaaa

SEQ ID NO: 10 - protein, 11 aa, Artificial Sequence
  Description of Artificial Sequence Peptide
LENGAIFVASG

(19) United States
(12) Patent Application Publication
(10) Pub. No.: US 2005/0215766 A1
(43) Pub. Date: Sep. 29, 2005

(54) Allergen from mugwort pollen

(76) Inventors: Fatima Ferreira, Salzburg (AT); Gudrun Hubinger, Weyregg am Attersee (AT); Christof Ebner, Brunn am Gebirge (AT); Klaus Richter, Anif (AT)

Correspondence Address: REED SMITH LLP, 1301 K STREET, N.W., SUITE 1100 EAST TOWER, WASHINGTON, DC, 20005, US

(21) Appl. No.: 10/517,052

(22) PCT Filed: Jun. 3, 2003
(86) PCT No.: PCT/EP03/05780

§ 371 Date: Dec. 3, 2004

(30) Foreign Application Priority Data
Jun. 4, 2002 (EP) 02012302.2

(51) Int. Cl. C07K 14/415; C07H 21/04

(52) U.S. Cl. 530/370; 536/23.6

(57) ABSTRACT

The present invention relates to a 40.9 kDa allergen from mugwort pollen, to polypeptides derived therefrom and polynucleotides encoding the same. The invention further pertains to antibodies directed against the allergen and to the use of the materials described for the manufacture of a medicament for the treatment or prevention of an allergic disorder.

Pollen grains originating from weeds, in particular those belonging to the very extensive plant family Compositae or Asteraceae, are common allergen sources throughout the world. The three most important wind-pollinated genera in this family are Ambrosia (ragweed), Parthenium (feverfew), and Artemisia vulgaris (mugwort). In late summer and autumn, pollen of mugwort is one of the main causes of allergic reactions in central Europe and parts of Asia (J. Charpin, R. Surinyach, and A. W. Frankland (1974) Atlas of European Allergenic Pollens (Paris: Sandoz)). Approximately 10% of patients suffering from pollinosis are sensitized by mugwort pollen. Ragweed pollen represents the major source of allergenic proteins in the United States and Canada, with a prevalence of about 50% in atopic individuals (L. P. Boulet, H. Turcotte, C. Laprise, C. Lavertu, P. M. Bedard, A. Lavoie, and J. Hebert (1997) Comparative degree and type of sensitization to common indoor and outdoor allergens in subjects with allergic rhinitis and/or asthma. Clin Exp Allergy 27, 52). In Europe, ragweed allergy is not a major cause of pollinosis, but it is rapidly increasing. Ragweed was first introduced into Europe at the beginning of last century and was limited to Hungary. During the Second World War it was introduced in France, thereafter gradually expanded and is now abundant in the Rhone valley (France), northern Italy, eastern parts of Austria, Hungary and Bulgaria (G. D'Amato, F. T. Spieksma, G. Liccardi, S. Jager, M. Russo, K. Kontou-Fili, H. Nikkels, B. Wuthrich, and S. Bonini (1998) Pollen-related allergy in Europe. Allergy 53, 567).

Nowadays, it is widely accepted that recombinant allergens represent promising tools for diagnosis and therapy of Type I allergy. The value of these molecules for diagnosis has been evaluated in detail and a panel of recombinant allergens is already available for routine diagnosis of certain inhalant allergies, including tree (birch)- and grass-pollen allergies (R. Valenta et al. (1992) Int Arch Allergy Immunol 97, 287; O. Scheiner, and D. Kraft (1995) Allergy 50, 384; D. Kraft et al. (1998) Allergy 53, 62; D. Kraft et al. (1999) Int Arch Allergy Immunol 118, 171) but not for weed pollen-allergy. Two major allergenic compounds of ragweed pollen known as antigens E (Amb a 1) and K (Amb a 2) were characterized and shown to be acidic proteins of about 38,000 Da (T. P. King (1980) Allergy 35, 187). cDNA cloning of Amb a 1 and Amb a 2 revealed that these allergens are highly homologous to each other and belong to the family of pectate lyase proteins (T. Rafnar et al. (1991) J Biol Chem 266, 1229; B. L. Rogers et al. (1991) J Immunol 147, 2547). Presently, no information about the complete structure and no molecular cloning data of any mugwort pollen allergen have been published. The only exception is a mugwort pollen allergen designated Art v 1 which is disclosed in WO99/49045. Art v 1 is not related to Amb a 1 or to any other characterized ragweed allergen. Several reports demonstrated that ragweed and mugwort pollen share common allergenic structures, i.e. antigens recognized by cross-reactive IgE antibodies in different allergen sources (C. Fernandez et al. (1993) J Allergy Clin Immunol 92, 660; R. Hirschwehr et al. (1998) J Allergy Clin Immunol 101, 196; H. S. Park et al. (1994) J Korean Med Sci 9, 213). IgE recognition of such common antigens will trigger allergic reactions in sensitized patients after contact with different allergen sources. Therefore, the characterization of mugwort pollen allergens and identification of common allergenic structures in ragweed pollen will provide basic information for the selection and production of recombinant allergens that are sufficient for the diagnosis and therapy of weed pollen-allergy.

It is therefore an object of the present invention to identify an allergen from mugwort pollen which might trigger type I allergy.

The inventors succeeded in isolating four bona fide and complete cDNA clones corresponding to a 40.9 kDa mugwort (Artemisia vulgaris) pollen allergen. The amino acid sequence of the novel 40.9 kDa mugwort pollen allergen is shown in SEQ ID NO:1.

The invention therefore relates to a polypeptide comprising an amino acid sequence which has an identity of at least 70% to the amino acid sequence as shown in SEQ ID NO:1. Preferably the polypeptide comprises an amino acid sequence which has an identity of at least 75%, more preferably an identity of at least 80%, even more preferably an identity of at least 85%, still more preferably an identity of at least 90%, most preferably an identity of at least 95% to the amino acid sequence as shown in SEQ ID NO:1. In a particular embodiment, the polypeptide comprises the amino acid sequence as shown in SEQ ID NO:1. As used herein, the term polypeptide refers to proteins and/or peptides having a length of at least 7 amino acids.

The degree of identity of an amino acid sequence to SEQ ID NO:1 is determined by comparing said amino acid sequence and SEQ ID NO:1 using the program “BLAST 2 SEQUENCES (blastp)” (Tatusova et al. (1999) FEMS Microbiol Lett. 174, 247-250) with the following parameters: Matrix BLOSUM62; Open gap 11 and extension gap 1 penalties; gap x_dropoff 50; expect 10.0 word size 3; Filter: none. According to the invention the sequence comparison covers at least 100 amino acids, preferably at least 200 amino acids, more preferably at least 300 amino acids, most preferably at least 350 amino acids.

The invention further relates to a polypeptide comprising a fragment of at least 18 amino acids of the amino acid sequence as shown in SEQ ID NO:1. Such polypeptide comprises at least 18 consecutive amino acids of the sequence as shown in SEQ ID NO:1. Preferably, the length of the fragment is at least 21 amino acids, more preferably at least 25 amino acids, even more preferably at least 35 amino acids, most preferably at least 50 amino acids of the amino acid sequence as shown in SEQ ID NO:1.

Another aspect of the invention is a polypeptide consisting of a fragment of at least 7 amino acids of the amino acid sequence as shown in SEQ ID NO:1. Preferably, the length of said polypeptide is at least 10 amino acids, more preferably at least 15 amino acids, even more preferably at least 25 amino acids, most preferably at least 35 amino acids. In further embodiments, the polypeptide of the invention consists of at least 8 or at least 9 or at least 11 or at least 12 or at least 13 or at least 14 or at least 18 or at least 21 or at least 30 contiguous amino acids from the amino acid sequence as shown in SEQ ID NO:1. In further aspects, the length of the polypeptide is not greater than 100 or 50 or 30 amino acids. Polypeptides consisting of at least 7 amino acids can be recognized by T cells (T cell epitopes).

Yet another aspect of the invention is a polypeptide comprising a fragment of the amino acid sequence as shown in SEQ ID NO:1 wherein said fragment is capable of binding to IgE antibodies from an individual being allergic against mugwort pollen. The term “IgE antibodies” means an antibody preparation which is obtainable by per se known processes. In susceptible humans, exposure to allergens leads to an immediate type (IgE-mediated) of allergic response, which is a two-step process:
(1) On the first exposure, allergenic proteins induce IgE synthesis by B-cells (Vercelli and Geha, 1989, J. Allergy Clin. Immunol. 9: 75-83). These specific IgE antibodies then bind to the surfaces of mast cells and basophils via high-affinity Fcε receptors (FcεRI).
(2) On subsequent exposure, allergens bind and crosslink these specific IgE antibodies leading to the release of pre-formed and newly synthesized inflammatory mediators (e.g. histamine) and chemotactic substances (e.g. platelet-activating factor).

In step (1), the production of allergen-specific IgE by B lymphocytes requires the “help” of T lymphocytes (Vercelli and Geha, 1989, J. Allergy Clin. Immunol. 9: 75-83), which are activated by linear peptide fragments of the allergen. These peptides are created by antigen-presenting cells through antigen processing and are displayed at the cell surface with molecules of the major histocompatibility complex (MHC), where they become available for recognition and binding to the T cell receptor (Schwartz, 1985, Annu Rev. Immunol. 3: 237-255; Rothbart et al., 1989, Int. Immunol. 1: 479-486). In step (2), allergen-specific IgE antibodies produced by B cells circulate and bind to FcεRI receptors on mast cells and basophils, thereby serving as the receptor for the allergen. Cross-linking of these cell surface-bound IgE antibodies by allergen represents the signal for the release of preformed and newly synthesized inflammatory mediators and chemotactic substances, leading to the typical allergic inflammatory reaction.

An individual being allergic against mugwort pollen is an individual that displays specific IgE antibodies recognizing mugwort pollen proteins and have typical allergic symptoms (IgE-mediated) when exposed to mugwort pollen. Such allergic reactions can also be triggered by exposure to homologous antigens present in other allergen sources. This phenomenon is defined as cross-reactivity. The selection of patients allergic to mugwort pollen was done according to typical case history, positive skin prick test and radioallergosorbent test “RAST”>3.5.

A polypeptide is capable of binding to an antibody if its affinity to the antibody is significantly higher than that of a reference composition which does not bind to the antibody. The binding of specific IgE antibodies to a particular polypeptide or allergen can be determined by various methods, e.g. RAST, immunoblot, ELISA, using as reference sera from healthy non-allergic individuals (according to case history, negative skin prick test, negative RAST), who do not display allergen-specific IgE antibodies.

In one embodiment of the invention the polypeptide is capable of binding to IgE antibodies from an individual being allergic against ragweed pollen. The present invention makes it possible to identify allergenic structures which are common to mugwort pollen and ragweed pollen. This information is useful for the selection and production of recombinant allergens that are sufficient for the diagnosis and therapy of weed pollen-allergy. Peptides and polypeptides containing such common allergenic structures are encompassed by the present invention.

During purification of natural Amb a 1 trypsin-like pollen protease cleaves this allergen. Thus, purification often results in the isolation of a full-length 38 kDa allergen plus two fragments of 26 and 12 kDa molecular weight, designated alpha (amino acids 187 to 396, numbering including signal peptide) and beta (amino acids 43 to 180, numbering including signal peptide), respectively. All chains are still highly reactive with polyclonal rabbit antibodies and human IgE from allergic patients. It is expected that proteolytic fragments of the natural 40.9 kDa mugwort allergen corresponding to these chains would also bind IgE antibodies from allergic patients. The first one corresponding to beta chain spans amino acids 21 to 180 and the second one corresponding to alpha chain spans amino acids 181 to 396, both numberings referring to SEQ ID NO:1. Both chains are expected to have epitopes which are also present in the Amb a 1 chains resulting in the same IgE reactivity. The invention relates to a polypeptide comprising a fragment of the sequence as shown in SEQ ID NO:1 wherein said fragment consists of amino acids 21 to 180 or 181 to 396 of the sequence as shown in SEQ ID NO:1.

In one embodiment, the polypeptide of the invention is not a polypeptide consisting of the amino acid sequence LENGAIFVASG (SEQ ID NO:10).

According to a preferred embodiment, the polypeptides of the invention are isolated polypeptides, i.e. they are in an essentially pure form. “Essentially pure” means that by separation of the polypeptides from other compounds the polypeptides are at least 75% pure, preferably at least 90% pure, more preferably at least 95% pure. The purity of polypeptides can be determined by the skilled person using techniques known in the field, e.g. by SDS-PAGE followed by protein staining. High Performance Liquid Chromatography (HPLC) and mass spectrometry are methods suitable for analyzing polypeptides and short peptides as well.

The polypeptides of the invention can be prepared in various ways. The polypeptides may be prepared by chemical synthesis, preferably by applying solid phase methods. Methods of chemical peptide synthesis are known to the skilled person (see, e.g., Merrifield, J. Am. Chem. Soc. 85:2149, 1963; Stewart et al., “Solid Phase Peptide Sythesis” (second edition), Pearce Chemical Co., Rockford, Ill., 1984 and Bayer and Rapp Chem. Pept. Prot. 3:3 (1986); and Atherton et al., Solid Phase Peptide Synthesis: A Practical Approach, IRL Press, Oxford, 1989). These methods are particularly suited for the preparation of short polypeptides having a length of 7 to 50 amino acids. Longer polypeptides may be prepared by chemically or enzymatically linking peptide fragments which have been prepared by chemical synthesis. The polypeptides of the invention may also be prepared by expression of DNA sequences in host cells. These methods are described in more detail below.

The present invention also concerns various polynucleotides. The polynucleotides may be single or double stranded DNA molecules, RNA molecules or nucleic acids which can be derived therefrom. According to a first aspect, the polynucleotides of the invention encode the amino acid sequence as shown in SEQ ID NO:1. Due to the degeneracy of the genetic code, many different polynucleotides can be envisaged which encode the amino acid sequence as shown in SEQ ID NO:1.

According to a second aspect, the polynucleotides of the invention encode a polypeptide according to the invention as described supra.

The polynucleotides described herein can be used to identify similar sequences in any variety or type of mugwort and, thus, to identify or isolate sequences which have sufficient homology to hybridize to, for example, DNA from mugwort pollen. This can be carried out, for example, under conditions of low stringency; those sequences which have sufficient homology (generally greater than 40%) can be selected for further assessment. Alternatively, high stringency conditions can be used. In general, high stringency conditions are selected to be about 5° C. to about 10° C. lower than the thermal melting point (T_{M}) for the specific sequence at a defined ionic strength and pH. The T_{M} is the temperature (under defined ionic strength and pH) at which 50% of the target sequence hybridizes to a perfectly matched probe. Typical high stringency conditions are those in which the salt concentration is up to about 15 mM Na⁺ (0.1×SSC) at pH 7, and the temperature is at least about 60° C.

In this manner, DNA of the present invention can be used to identify, in other types of weed pollen sequences encoding peptides having amino acid sequences similar to that of the 40.9 kDa protein described herein and, thus, to identify allergens in such other types of weed pollen. Thus, the present invention includes not only the 40.9 kDa protein and other mugwort allergens encoded by the present DNA sequences, but also other weed pollen allergens encoded by DNA which hybridizes to DNA of the present invention.

Therefore, according to a third aspect, the polynucleotides of the invention comprise a nucleotide sequence which has an identity of at least 75% to the nucleotide sequence as shown in SEQ ID NO:2. Preferably, the identity is at least 80%, more preferably at least 90%, most preferably at least 95%. The degree of identity of a polynucleotide sequence to SEQ ID NO:2 is determined by comparing said polynucleotide sequence and SEQ ID NO:2 using the program “BLAST 2 SEQUENCES (blastn)” (Tatusova et al. (1999) FEMS Microbiol Lett. 174, 247-250) with the following parameters: Reward for a match 1; Penalty for a mismatch −2; Open gap 5 and extension gap 2 penalties; gap x_dropoff 50; expect 10.0; word size 11; Filter: None. According to the invention the sequence comparison covers at least 300 nucleotides, preferably at least 600 nucleotides, more preferably at least 900 nucleotides, most preferably at least 1100 nucleotides.

In one embodiment the polynucleotide comprises the nucleotide sequence as shown in SEQ ID NO:2 or the nucleotide sequence as shown in SEQ ID NO:3. In another embodiment, the polynucleotide of the invention comprises at least 100, preferably at least 300, more preferably at least 600, most preferably at least 1000 contiguous nucleotides of the sequence as shown in SEQ ID NO:2 or SEQ ID NO:3.

The invention also covers polynucleotides which are degenerate to the polynucleotides described supra. The respective complementary strands of the polynucleotides disclosed are encompassed by the present invention.

The polynucleotides of the present invention preferably are isolated polynucleotides. The polynucleotides preferably are essentially pure, i.e. they are at least 80% pure, more preferably at least 90% pure, most preferably at least 95% pure. The polynucleotides according to the invention can be prepared in various ways (see, e.g., Glick and Pasternack, Molecular Biotechnology, Principles and Applications of recombinant DNA, ASM Press, Washington D.C. 1994; Itakura et al. Annu. Rev. Biochem. 53:323-56, 1984 and Climie et al., Proc. Natl. Acad. Sci. USA 87:633-7, 1990). They may be synthesized by chemical methods usually employed in oligonucleotide synthesis. PCR based methods can be used to synthesize the polynucleotides, in particular longer polynucleotides (see, e.g. Lee et al., Nucleic Acid Amplification Technologies, Eaton Publishing, Natick, Mass. 1997; McPherson, M. J. et al. PCR—A Practical Approach, Vol. 1 and 2, IRL Press, Oxford 1996). Longer polynucleotides can also be prepared by chemically or enzymatically linking fragments which have been synthesized using chemical methods.

Another aspect of the invention is a plasmid or a vector comprising a polynucleotide according to the invention. The plasmids may contain regulatory sequences which facilitate replication of the plasmids or transcription and/or translation of encoded sequences. Examples of such sequences are promotors, terminator sequences, etc. The plasmids may also contain nucleotide sequences encoding amino acid sequences which facilitate the purification of encoded polypeptides upon expression in a host cell. Examples of such sequences are a 6×His tag, a FLAG tag, and sequences encoding bacterial proteins such as GST. Purification can be achieved by affinity chromatography using antibodies directed against the respective tag sequences or bacterial sequences. Plasmids and vectors containing such tag sequences or bacterial sequences are known to the skilled person.

The invention further pertains to a cell containing a plasmid or a vector and/or a polynucleotide according to the present invention. The cells may be selected from plant cells, bacterial cells, yeast cells and other cells. Preferred are cells which allow for the expression of the genes encoded by the polynucleotides of the invention. Most preferably, the cells are E. coli cells. The plasmid, vector and/or polynucleotide according to the present invention may be introduced into the cells by techniques known per se, e.g. transformation, transfection etc. The cells can contain the nucleic acid molecules only transiently or stably integrated into their genome. In particular in the latter case, the nucleic acid molecules may be truncated or fragmented due to the process of integration into the genome. Cells containing such truncated or fragmented versions of the nucleic acid molecules are within the scope of the present invention.

The cells can be used for the expression and purification of the polypeptides of the invention. The invention therefore relates to a process for the preparation of a polypeptide according to the invention comprising the step of culturing the cells described in the present invention under conditions appropriate for the expression of the polypeptide and optionally subsequently recovering the polypeptide. The cells are preferably cultured in a liquid medium under agitation. If appropriate, gene expression is induced by an inducing agent, e.g. IPTG. Techniques for manipulating cloned DNA molecules and introducing exogenous DNA into a variety of host cells, techniques for transforming these hosts and techniques for expressing foreign DNA sequences cloned therein are well known in the art and disclosed in, e.g., Sambrook et al., Molecular Cloning: A Laboratory Manual, second edition, Cold Spring Harbour Laboratory Press, Cold Spring Harbour, New York 1989; and Ausubel et al., eds., Current Protocols in Molecular Biology, John Wiley and Sons, Inc., New York 1987.

Following cultivation the cells may be opened using established methods and the polypeptide may recovered by affinity chromatography. Other known methods of protein purification can be employed. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. In a preferred embodiment, the polypeptide of the invention is isolated by a method comprising an affinity chromatography step. According to this embodiment, one or more antibodies directed against the polypeptide of the invention may be immobilized to a solid support. Preferably, the antibody specifically recognizes the polypeptide of the invention. Methods for binding antibody molecules to support media are well known in the art. Selection of a particular method is a matter of routine design and is determined in part by the properties of the chosen support.

Alternatively, immobilized metal ion adsorption chromatography can be used to purify histidine-rich proteins, including those comprising polyhistidine tags. Briefly, a gel is first charged with divalent metal ions to form a chelate. Histidine-rich proteins will be adsorbed to this matrix with differing affinities, depending on the metal ions used, and will be eluted by competitive elusion, lowering the pH, or use of strong chelating agents. Within additional embodiments of the invention, a fusion of the polypeptide of interest and an affinity tag may be constructed to facilitate purification. Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. Methods of protein purification are described, e.g., in Deutscher, M. P. Protein Purification, Academic Press, New York 1990; Scopes, R. K. Protein Purification, Springer Verlag, Heidelberg 1994; Doonan, S. Protein Purification Protocols. Humana Press, 1996.

Polypeptides of the present invention can be used, for example, as “purified” allergens. Such purified allergens are useful in the standardization of allergen extracts which are key reagents for the diagnosis and treatment of ragweed allergy. Furthermore, by using peptides based on fragments of the amino acid sequence as shown in SEQ ID NO:1, anti-peptide antisera or monoclonal antibodies can be made using standard methods. These sera or monoclonal antibodies, directed against the 40.9 kDa protein of the invention, can be used to standardize allergen extracts.

Another aspect of the invention is an antibody which is capable of binding to a polypeptide according to the present invention. The antibody may be a polyclonal or a monoclonal antibody, monoclonal antibodies, however, are preferred. Antibodies directed against the polypeptide of the invention may be prepared in a known manner, see, e.g., Harlow and Lane 1988 “Antibodies: A Laboratory Manual”; Cold Spring Harbour Laboratory. In a particular embodiment the antibody is in an essentially pure form, i.e. at least 80% pure, preferably at least 90% pure. In the case of polyclonal antibodies, the antibody composition contains many different species of antibody molecules.

The antibody may be specific for the polypeptide comprising the amino acid sequence as shown in SEQ ID NO:1. In this embodiment, the antibody does not bind to anyone of the polypeptides Amb a I.1, Amb a I.2, Amb a I.3 and Amb a 2 from ragweed pollen.

In another embodiment, however, the antibody is capable of binding to one or several (e.g., two, three or four) of the polypeptides Amb a I.1, Amb a I.2, Amb a I.3 and Amb a 2 from ragweed pollen.

Antibodies according to the invention can be used to isolate additional components of mugwort allergens which can be used for further definition of the characteristics of this family of allergens. Furthermore, anti-peptide sera or monoclonal antibodies directed against the polypeptides of the invention can be used to standardize and define the content of skin test reagents.

The invention further concerns a pharmaceutical composition comprising a polypeptide and/or a polynucleotide and/or an antibody according to the present invention.

The materials described herein, as well as compositions containing these materials, can be used in methods of diagnosing, treating and preventing mugwort allergy. Another aspect of the invention therefore is the use of a polypeptide and/or a polynucleotide and/or an antibody according to the present invention for the preparation of a medicament for the treatment or the prevention or the diagnosis of an allergic disorder.

The medicament is preferably administered to an individual to be desensitized.

Through use of the polypeptides of the present invention, allergen preparations of consistent, well-defined composition and biological activity can be made and administered for therapeutic purposes (e.g., to modify the allergic response of a mugwort-sensitive individual to a mugwort pollen). Such polypeptides or modified versions thereof may, for example, modify B-cell response to a mugwort allergen, T-cell response to a mugwort allergen or both responses. Purified allergens can also be used to design modified derivatives or analogues which are more useful in immunotherapy than are the unmodified naturally-occurring peptides.

High doses of allergens generally produce the best symptom relief. However, many people do not tolerate high doses of allergens because of allergic reactions to the allergens. Modification of naturally-occurring allergens can be designed in such a manner that modified polypeptides which have the same or enhanced therapeutic properties as the corresponding naturally-occurring allergen but have reduced side effects, especially reduced anaphylactic reactions, can be produced. These can be, for example, a polypeptide of the present invention or a modified analogue (e.g., a polypeptide in which the amino acid sequence has been altered to modify immunogenicity and/or reduce allergenicity or to which a component has been added for the same purpose). For example, the polypeptides can be modified using the polyethylene glycol method of A. Sehon and co-workers. Short segment deletion or amino acid substitutions of the polypeptide sequence results in a weakened (weaker or no) antibody binding. This modification leads to low IgE binding and is very useful in immunotherapy due to less side effects.

Administration of the peptides of the present invention to an individual to be desensitized can be carried out using known techniques. A peptide or combination of different peptides can be administered to an individual in a composition which includes, for example, an appropriate buffer, a carrier and/or an adjuvant. Such compositions will generally be administered by injection, oral administration, inhalation, transdermal application or rectal administration. Using the structural information now available, it is possible to design a mugwort pollen polypeptide which, when administered to a mugwort-sensitive individual in sufficient quantities, will modify the individual's allergic response to a mugwort allergen. This can be done, for example, by examining the structures of the mugwort proteins, producing peptides to be examined for their ability to influence B-cell and/or T-cell responses in mugwort-sensitive individuals and selecting appropriate epitopes recognized by the cells. Synthetic amino acid sequences which mimic those of the epitopes and which are capable of down regulating allergic response to mugwort allergen can also be used.

Polypeptides or antibodies of the present invention can also be used for detecting and diagnosing mugwort allergy. For example, by combining blood or blood products obtained from an individual to be assessed for sensitivity to mugwort allergen with an isolated allergenic peptide of mugwort pollen, under conditions appropriate for binding of components (e.g., antibodies, T cells, B cells) in the blood with the polypeptide and determining the extent to which such binding occurs.

It is now also possible to design an agent or a drug capable of blocking or inhibiting the ability of mugwort allergens to induce an allergic reaction in mugwort-sensitive individuals. Such agents could be designed, for example, in such a manner that they would bind to relevant anti-mugwort IgEs, thus preventing IgE-allergen binding and subsequent mast cell degranulation. Alternatively, such agents could bind to cellular components of the immune system, resulting in suppression or desensitization of the allergic response to mugwort allergens. A non-restrictive example of this is the use of appropriate B- and T-cell epitope peptides, or modifications thereof, based on the cDNA/polypeptide structures of the present invention to suppress the allergic response to mugwort allergens. This can be carried out by defining the structures of B- and T-cell epitope peptides which affect B- and T-cell function in in vitro studies with blood cells from mugwort-sensitive individuals.

Finally, the invention relates to kits which are useful for the diagnosis, the treatment and/or the prevention of an allergic disorder comprising a polypeptide and/or a polynucleotide and/or an antibody according to the present invention. The materials described herein, as well as compositions and kits containing these materials, can be used in methods of diagnosing, treating and preventing mugwort allergy.

The present invention is based on a recombinant DNA molecule and fragments thereof coding for a 40.9 kDa mugwort (Artemisia vulgaris) pollen allergen, which shows sequence homology to Amb a 1, a major allergen of ragweed (Ambrosia artemisiifolia) pollen, on a method for the isolation of Art v 1 molecules or fragments thereof, and on an expression vector and a host cell transformed to express the nucleic acid sequences of the invention. The present invention further relates to methods of diagnostics and therapy of weed pollen-allergic patients. Because the 40.9 kDa mugwort allergen shows sequence homology to Amb a 1, this mugwort protein might represent a cross-reactive allergen and could be used for diagnosis and therapy of not only mugwort pollen allergy, but of weed pollen-allergy in general.

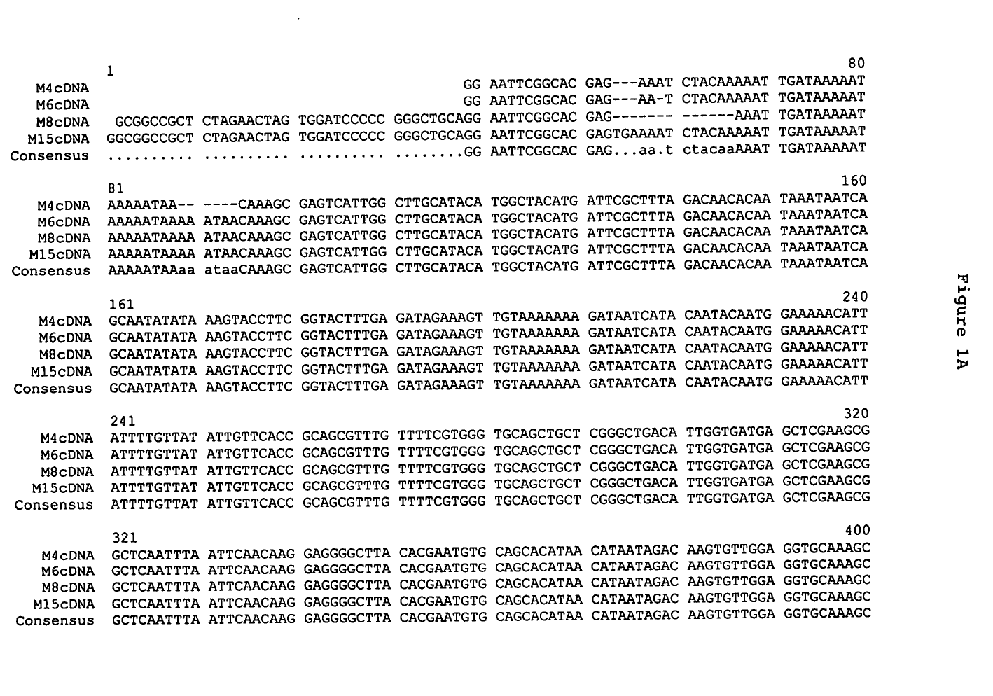

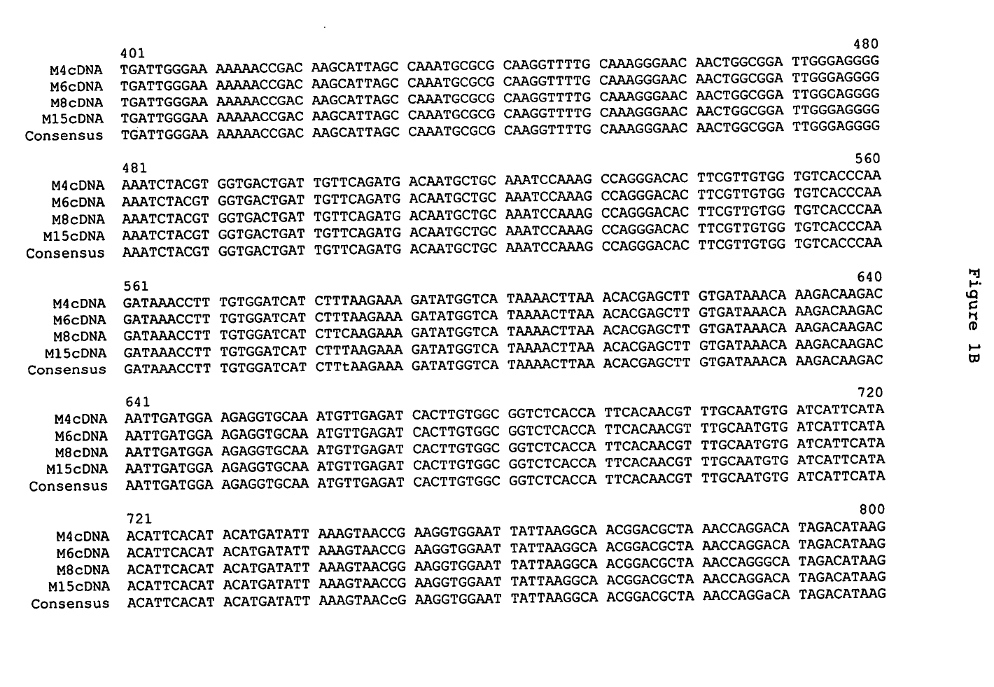

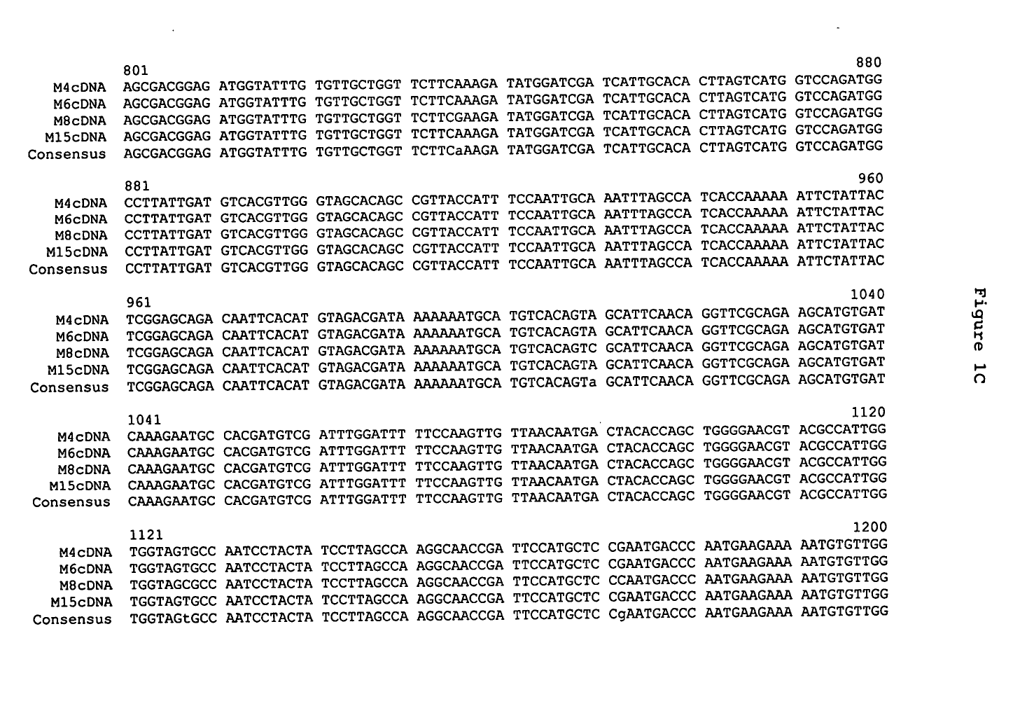

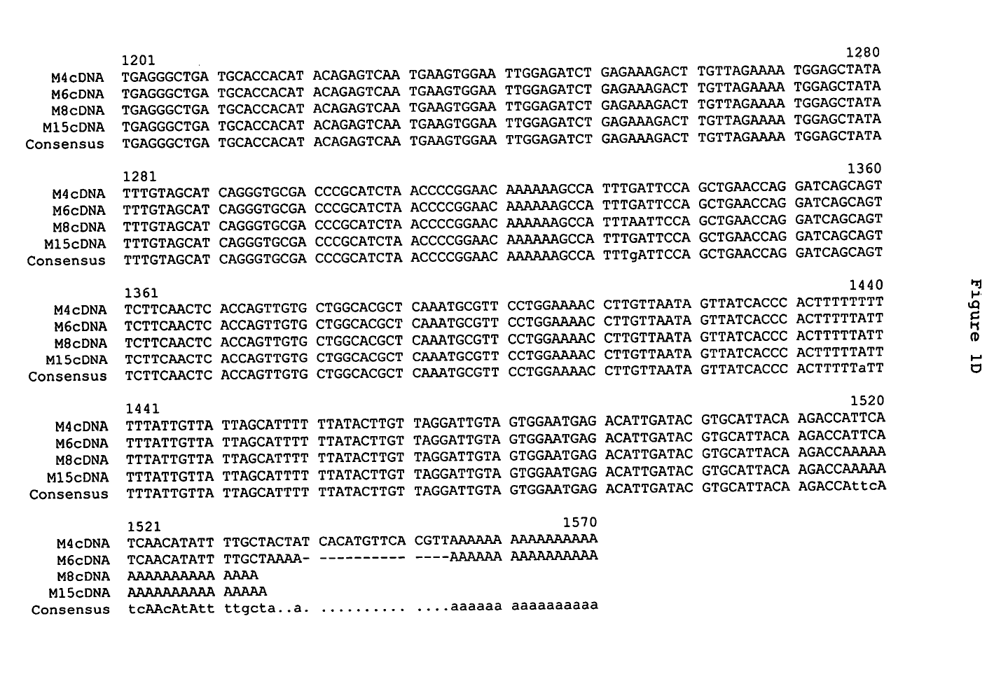

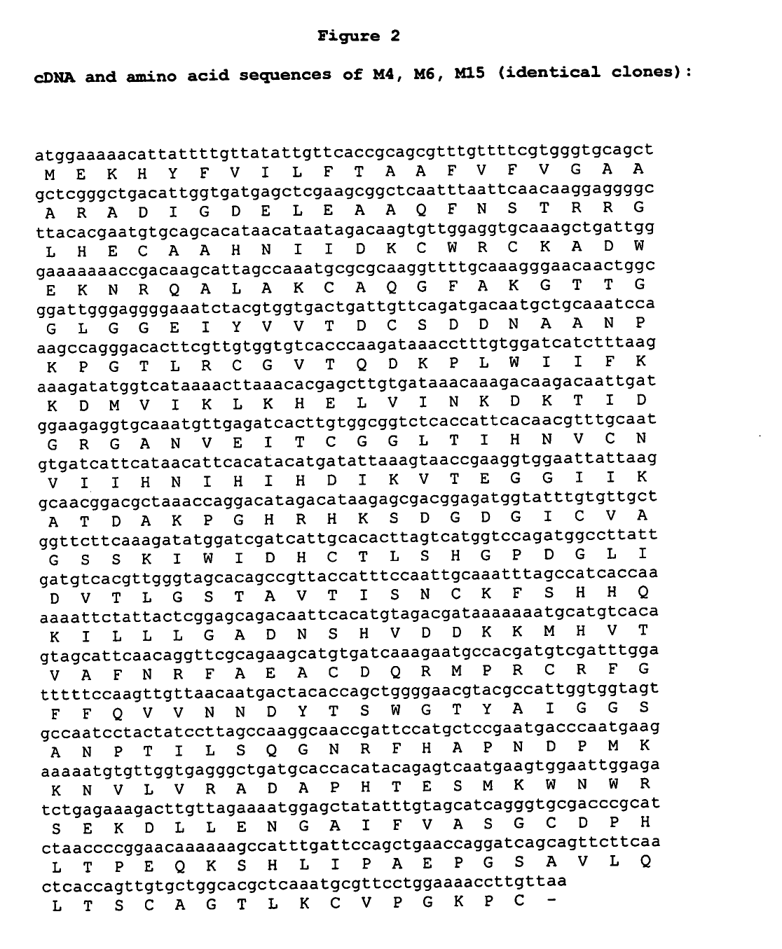

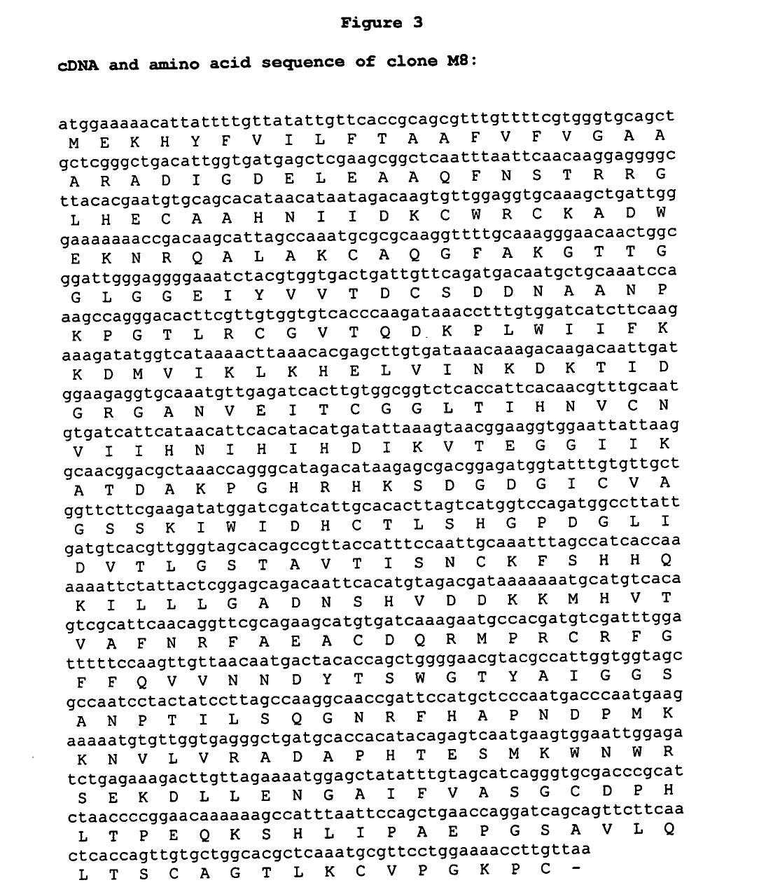

The present application describes the isolation of four bona fide and complete cDNA clones corresponding to a 40.9 kDa mugwort (Artemisia vulgaris) pollen allergen. The cDNA nucleotide sequences and derived amino acid sequences of the clones M4, M6, and M15 were identical (See FIGS. 1 and 2). Clone M8 differed from the other by 8 nucleotide substitutions, which did not lead to amino acid exchanges (See FIG. 3). The clones are complete in their 5′ ends because they contain the start AUG codon in a typical eukaryotic context and some sequence upstream of the start codon. The clones are complete in the 3′ region because they contain 90-120 nucleotides after the stop codon followed by the polyA⁺-tail. The sequences outside the open reading frame are not shown in FIGS. 2 and 3. The alignments of the nucleotide sequences of clones M4, M6, M8 and M15 are shown in FIG. 1A-D. The nucleotide sequence of the coding region and the deduced amino acid sequence of clones M4, M6 and M15 are shown in FIG. 2. The nucleotide sequence of the coding region and the deduced amino acid sequence of clone M8 are shown in FIG. 3.

FIGS. 1A, 1B, 1C and 1D show an alignment of the nucleotide sequences of clones M4, M6, M8 and M15 (SEQ ID NO:6, 7, 8, and 9). Capitals in the consensus sequence indicate identical nucleotides in the four clones.

FIG. 2 shows the nucleotide sequence of the coding region (SEQ ID NO:2 plus stop codon) and the deduced amino acid sequence (SEQ ID NO:1) of clones M4, M6 and M15.

FIG. 3 shows the nucleotide sequence of the coding region (SEQ ID NO:3 plus stop codon) and the deduced amino acid sequence (SEQ ID NO:1) of clone M8.

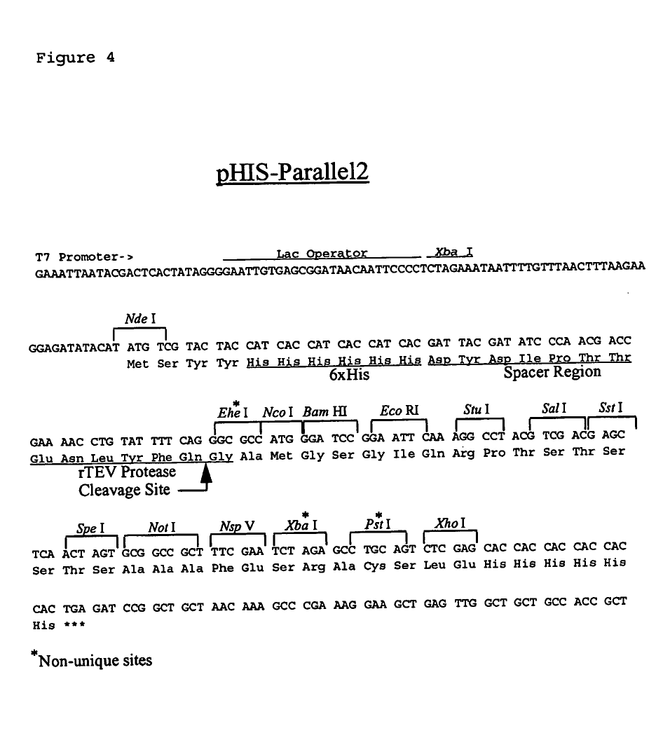

FIG. 4 shows the structure of the multiple cloning site of plasmid “pHis-Parallel2” as used in Example 2. The amino acids encoding a 6×His tag, a spacer region and a protease cleavage site are also indicated.

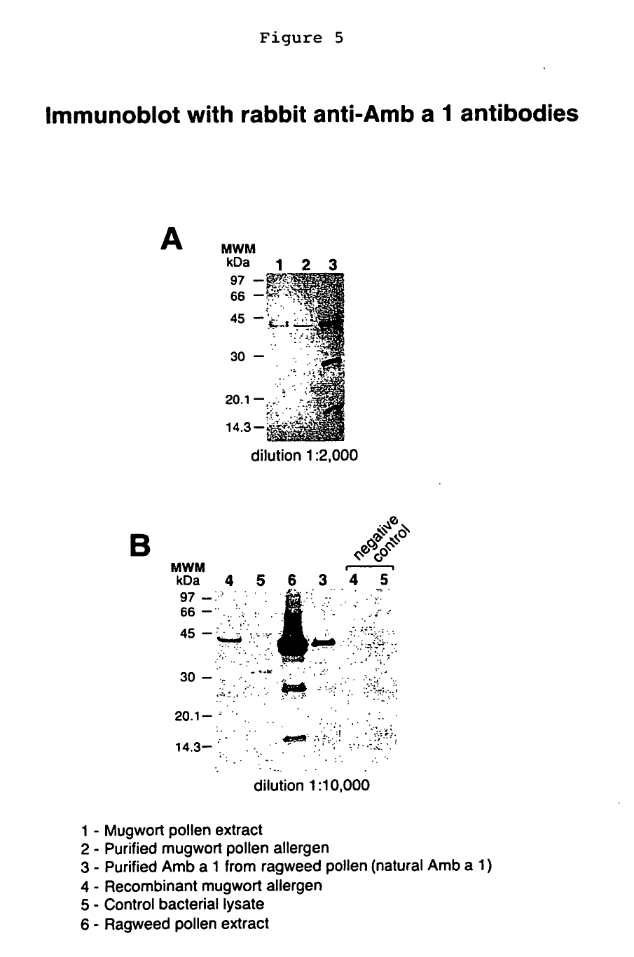

FIG. 5A shows Mugwort pollen extract, purified mugwort pollen allergen and purified Amb a 1 from ragweed pollen separated by gel electrophoresis followed by Coomassie staining. FIG. 5B shows an immunoblot with rabbit anti-Amb a 1 antibodies (See Examples 3 and 4).

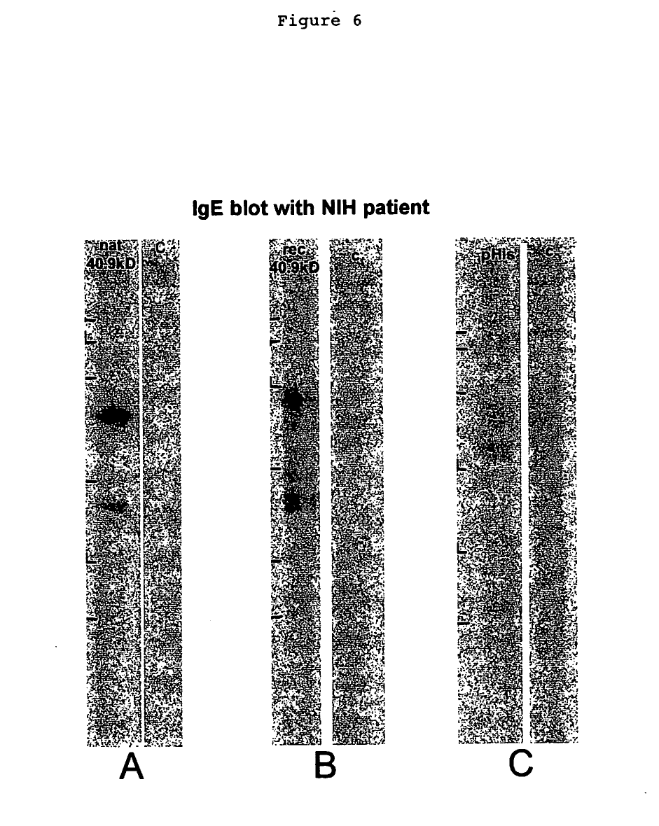

FIG. 6 shows an immunoblot with IgE antibodies (See Examples 3 and 4).

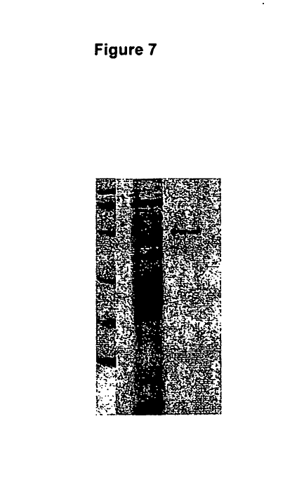

FIG. 7 shows a Coomassie-stained gel after SDS-PAGE. Lane 1 (left): Molecular weight marker RPN 756 (Amersham Biosciences); lane 2 (middle): Mugwort pollen extract; lane 3 (right): purified recombinant 40.9 kDa mugwort pollen protein, ˜3 μg (see Example 5).

The present invention will be further illustrated by the following examples which are not intended to be limiting.

### EXAMPLE 1

### Immunoscreening of a Mugwort Pollen cDNA Library with Purified Rabbit anti-Amb a 1 Antibodies

The isolation of the cDNA clone coding for the 40.9 kDa mugwort pollen allergen was done in the following way:

The first step for the cDNA cloning of the 40.9 kDa allergen was the isolation of RNA from mugwort pollen, which turned out to be a very difficult procedure. Using several different standard procedures for RNA isolation, there were always pigments and other organic substances co-purifying with the RNA that inhibited enzymes used for the cDNA synthesis. However, after a series of trials it was possible to isolate mRNA sufficiently pure to construct a cDNA library, which was also done in the expression vector lambda ZAP.

Sera from rabbits immunized with Amb a 1 was kindly provided by Dr. Te Piao King, (Rockefeller University, USA). For screening the mugwort cDNA library we first purified Amb a 1-specific antibodies by affinity chromatography. For that purpose, 5 mg of Amb a 1 purified from ragweed pollen was coupled to CNBr-activated Sepharose (Pharmacia). After binding of the rabbit sera, the resin was washed and Amb a 1-specific antibodies eluted with 0.2 M glycine, pH 2.8. The antibodies were neutralized and dialyzed against PBS. With these purified rabbit anti-Amb a 1-antibodies 450.000 plaques of a mugwort pollen cDNA library constructed in lambda ZAP II phages (Stratagene, Calif., USA) were screened. In total, 13 positive clones were isolated and used for single clone in vivo excision of pBluescript phagemid from the Uni-ZAP XR vector. Four clones (M4, M6, M8, and M15) were selected for DNA sequence analysis, which was carried out by the “primer walking” technique using 5 primers including the flanking primers T7 und T3. Both strands were sequenced twice. The aligned nucleotide sequences of the four clones M4, M6, M8, and M15 are shown in FIGS. 1A-1D. The cDNA sequences of clones M4, M6 and M15 were identical (FIG. 2). The cDNA sequence in the coding region of M8 differed from the other 3 clones by 8 bases (FIG. 3). However, all four clones lead to the same protein with a calculated molecular weight of 40.9 kDa (FIGS. 2 and 3).

Finally, the sequence of the 40.9 kDa protein was used for similarity searches in the Database. Significant sequence homology was found to Amb a 1, a major allergen from ragweed pollen belonging to the pectate lyase family (63.9% identity).

The cDNA of the invention can now be obtained based on the sequence information as described herein. The cDNA can be cloned from a cDNA library using as a probe an oligonucleotide or polynucleotide which was prepared by chemical synthesis. Alternatively, the cDNA can be obtained by PCR using primers which are derived from SEQ ID NO:2. Such methods are known to the skilled person.

### EXAMPLE 2

### Cloning of the 40.9 kDa Mugwort Allergen (clone M4) into the Expression Plasmid pHIS-Parallel-2

The expression plasmid containing the 40.9 kDa mugwort allergen cDNA was constructed in the vector pHis-parallel 2 (see FIG. 4). For the cloning procedure two flanking cloning primers were constructed. The complete cDNA sequence was truncated at the 5′ end by 60 nucleotides coding for the putative signal peptide. In this way only the mature form of the protein was produced in E. coli.

The following primers were used: Mug-Nco-fwd 5′ GAG AGA GAC CAT GGC TCG GGC TGA CAT TGG TGA TGA GCT CG 3′ (SEQ ID NO:4) and Mug-Xho-rev 5′ GAG AGA GAC TCG AGT TAA CM GGT TTT CCA GGA ACG CAT TTG 3′ (SEQ ID NO:5) for PCR, Nco I and Xho I restriction sites were introduced at the 5′ and 3′ ends. PCR products were digested with Nco I and Xho I restriction enzymes and ligated to the respective sites of the vector pHis-parallel-2. The clone was analyzed by DNA sequencing.

### EXAMPLE 3

### Expression of the Recombinant 40.9 kDa Mugwort Allergen in E. coli

For the recombinant production of the 40.9 kDa mugwort allergen, competent E. coli strain BL21 (DE3, Stratagene, Calif., USA) was transformed with the expression plasmid pHis-parallel-2-M4 and selected on LB plates containing 100 mg/l ampicillin. A single transformant colony was picked and bacteria were grown in a 25 ml overnight pre-culture with LB medium containing ampicillin to an optical density of 0.6 at 600 nm. LB-amp medium was added to a volume of 250 ml. Isopropyl-β-D-galactopyranoside (IPTG) was added to a final concentration of 1 mM and incubation continued for 7 hours at 37° C. The bacterial cells were harvested by centrifugation and pellets were resuspended in 50 mM phosphate buffer pH8 containing 300 mM NaCl and 10 mM imidazole. Cells were then disrupted by three cycles of freezing in liquid nitrogen, followed by thawing at 37° C. After centrifugation at 5.500 rpm for 25 minutes, the supernatant was removed (soluble fraction) and 6 M urea was added to the pellet. After centrifugation, the supernatant was removed (insoluble fraction), and both soluble and insoluble fractions analyzed by SDS-PAGE. The recombinant 40.9 kDa mugwort protein was recovered in the insoluble fraction, which was then used for immunoblots with anti-Amb a 1 antibodies (FIG. 5B, lane 4) and with serum IgE from weed pollen-allergic patients (FIG. 6, lanes 3 and 4). As control, E. coli strain BL21 transformed with pHis-parallel-2 without an insert was processed as described above and the insoluble fraction used for the immunoblot experiments (FIG. 6, lanes 5 and 6).

FIG. 5A, lane 1 shows the mugwort pollen extract which contains all the proteins (allergens or nonallergens) released from the pollen.

Preparation of the Mugwort Pollen Extract:

150 mg of mugwort pollen grains (Allergon AB, Sweden) were mixed with 1 ml of water. The pollen suspension was gently agitated at room temperature for a 10 hour period. After centrifugation the pattern of proteins released from the mugwort pollen was analyzed by SDS-PAGE and Coomassie staining.

Lane 2 of FIG. 5A shows purified natural 40.9 kDa mugwort pollen allergen. The purification was done via immunoaffinity chromatography method. Purified rabbit anti Amb a 1 antibodies were coupled to a cyanogen bromide acivated sepharose CL4B (Pharmacia Biotech) and used for the purification of the natural 40.9 kDa mugwort protein from a filtered 10 hours pollen extract.

Lane 3 of FIG. 5A shows purified natural Amb a 1, a major allergen in the ragweed pollen. The purification was also done via immunoaffinity chromatography method. Purified rabbit anti Amb a 1 antibodies were coupled to a cyanogen bromide acivated sepharose CL4B (Pharmacia Biotech) and used for the purification of the natural Amb a 1 from a filtered 10 hours ragweed pollen extract including 10 mM protease inhibitor 1,10phenantroline.

### EXAMPLE 4

### Purification of the Natural 40.9 kDa Allergen from Mugwort Pollen

The natural 40.9 kDa allergen was purified from aqueous extract of mugwort pollen by immunoaffinity chromatography. Purified rabbit anti-Amb a 1 antibodies were coupled to CNBr-activated sepharose CL4B and used for the purification of the 40.9 kDa allergen. The purified natural 40.9 kDa allergen was then tested in immunoblots with anti-Amb a 1 antibodies (FIG. 5A, lane 2) and with IgE antibodies from allergic patients (FIG. 6, lanes 1 and 2).

A cross-reactivity is occurring between the US patient sensitized for ragweed and the purified natural and recombinant 40.9 kDa protein in mugwort. Therefore a positive signal can be observed in FIGS. 6A and 6B. It is expected that many more patients show a cross-reactivity between these two allergens.

### EXAMPLE 5

### Expression and Purification of Recombinant 40.9 kDa Mugwort Pollen Protein

The protein expression was carried out in E. coli BL21-DE3 hosting a pHis parallel 2 plasmid with inserted cDNA coding for the 40.9 kDa mugwort pollen protein.

The culture medium (1% Peptone, 0.5% Yeast Extract, 0.5% NaCl, 10 g Glycerin/1 L, 2 mM MgSO₄; 20 g (NH₄)₂SO₄, 10 mM PO₄, pH adjusted to 7.4) was inoculated with 3% of a saturated overnight culture, grown in the culture medium with 150 μg/mL penicillin G (Biochemie, Kundl, Austria). Fermentation was carried out in a 10 L Bioflow 3000 Fermenter (New Brunswick Scientific Co., Edison, N.J.) at 37° C. with 7% oxygen saturation 200-400 rpm agitation and induction with 0.4 mM IPTG at an optical density of 0.8.

Bacterial cells were harvested by centrifugation 5 hours post induction (20 g wet cell weight from 10 L culture) and resuspended in 100 mL lysis buffer (50 mM Tris base, 1 mM EDTA, 0.1% Triton X-100, pH non-adjusted, 5 mL/g cells). After addition of freshly dissolved lysozyme (100 μg/g cells) and incubation for 1 hour at room temperature, the cells were lysed by 3 freeze-thaw cycles at −70° C. and 37° C. The DNA was sheared with an ultraturax. Separation of the soluble fraction was done by centrifugation and the raw inclusion bodies were washed twice with 1% Triton X-100, 20 mM Tris-HCl pH 8.0, 1 mM EDTA, followed by 2 washes with 50% ethanol, 20 mM Tris-HCl pH 8.0. Purified inclusion bodies were dissolved in 500 mL 8 M urea, 0.5 M NaCl, 20 mM Tris HCl pH 8.0 and loaded on a 150 mL Chelating Cellufine Column (Millipore, Bedford, Mass., USA) pre-equilibrated with the same buffer, after charging it with 0.1M NiCl₂ according to the manufacturers instructions. All chromatography steps were carried out on a Biopilot FPLC system (Amersham Biosciences, Uppsala, Sweden). Bound protein was eluted with a linear imidazole gradient ranging from 0-500 mM. The purity of the fractions was analyzed by conventional SDS-PAGE. Due to impurity (high background) of the fractions containing the recombinant 40.9 kDa mugwort pollen protein, cation exchange chromatography under denaturing conditions was carried out.

A 180 mL column with the CEC matrix “Matrex Cellufine C-500” (Millipore) was equilibrated with 200 mL 100 mM NaAc pH 5.5. After washing the column with 400 mL binding buffer (5 M urea, 20 mM NaAc, 10 mM β-mercaptoethanol, 2 mM EDTA, pH 5.5) the protein solution was applied with a pH of 5.5.

Again, the fractions were analysed by SDS-PAGE and Coomassie staining. The fractions containing the protein of interest were pooled and prepared for the gel filtration step under denaturing conditions and a basic pH.

Gel filtration was performed in 6 M urea, 0.3 M NaCl, 30 mM Tris HCl pH 8.0, 2 mM EDTA, 10 mM β-mercaptoethanol on a Sephacryl S-200 HR (Amersham Biosciences, Uppsala, Sweden) column (dimensions 50×1000 mm).

Pure 40.9 kDa mugwort pollen protein fractions were concentrated and refolded by filtration through an Amicon ultrafiltration device (millipore) while adding 5 mM NH₄HCO₃. The overall yield was 4-6 mg protein.

FIG. 7, lane 3 shows the purified recombinant 40.9 kDa mugwort pollen allergen with His-tag (˜3 μg) after SDS-PAGE and Coomassie staining.

### EXAMPLE 6

### IgE Blots with Recombinant 40.9 kDa Mugwort Pollen Protein

Sera from different allergic or sensitized patients were tested for reactivity to the novel 40.9 kDa mugwort pollen allergen. The tests were carried out according to the following protocol:
preparative 15% SDS gels
load ˜80 μg purified recombinant 40.9 kDa protein (obtained, e.g., according to Example 5)
SDS-gel electrophoresis 100V, 2.5 hrs
Western blot: on a PVDF membrane 2 hrs, 300 mA
Incubation with 1^{st} antibody: human sera: dilute 1:10 in gold buffer for at least 6 hrs at RT
Wash step 3×10′ in gold buffer
Incubation with 2^{nd} antibody: Radiolabeled [¹²⁵I]-Anti-Human IgE (also referred as RAST), delivered by MedPro (Cat.# VE39020, 5.2 ml, 5 μCi)—1:40 in gold buffer, ON at RT
wash 3×10′ in gold buffer
place the phosphor-imager screen with the white site heading the membrane strips
expose screen in phosphoimager cassette 24-48 hrs

develop the screen in the phosphor-imager.

| | | |
|---|---|---|
| Blocking Buffer = | 7.5 | g Na₂HPO₄ |
| Gold buffer (1 L): | 1.0 | g NaH₂PO₄ |
| | 5.0 | g BSA |
| | 5.0 | ml Tween20 |
| Transfer buffer (per 5 L): | 15.13 | g Tris base |
| | 72.06 | g glycine |
| | 1 | L methanol absolute |

Add dH₂O

A serum was classified “IgE-positive” when the signal corresponding to the 40.9 kDa band was significantly above the background signal.

The Following Results were Obtained:

TABLE 1
Ragweed reference sera from NIH/USA (lane 1-3) and mugwort
allergic patients from Austria (lane 4-22): Blot I

| # | Patient | IgE-positive | IgE-negative |
|---|---|---|---|
| 1 | Human anti ragweed | | x |
| 2 | #3130 | x | |
| 3 | #3131 | | x |
| 4 | 180.212 | | x |
| 5 | 181.822 | | x |
| 6 | 492.788 | | x |
| 7 | 493.050 | x | |
| 8 | 493.087 | | x |
| 9 | 493.201 | | x |
| 10 | 493.021 | | x |
| 11 | 494.190 | | x |
| 12 | 180.912 | | x |
| 13 | 180.889 | x | |
| 14 | 180.682 | x | |
| 15 | 180.178 | x | |
| 16 | 493.647 | | x |
| 17 | 493.173 | x | |
| 18 | 493.12x | | x |
| 19 | 492.712 | | x |
| 20 | 494.102 | | x |
| 21 | 493.910 | | x |
| 22 | 493.885 | x | |

TABLE 2
Mugwort allergic patients from Austria (lane 23-44): Blot II

| # | Patient | IgE-positive | IgE-negative |
|---|---|---|---|
| 23 | 493.593 | x | |
| 24 | 493.510 | | x |
| 25 | 493.485 | x | |
| 26 | 493.428 | | x |
| 27 | 493.311 | x | |
| 28 | 494.169 | | x |
| 29 | 494.105 | | x |
| 30 | 492.395 | | x |
| 31 | 492.391 | x | |
| 32 | 183.011 | | x |
| 33 | 182.881 | x | |
| 34 | 182.729 | x | |
| 35 | 182.726 | x | |
| 36 | 182.484 | | x |
| 37 | 182.483 | x | |
| 38 | 182.333 | x | |
| 39 | 182.259 | | x |
| 40 | 182.083 | | x |
| 41 | 181.713 | | x |
| 42 | 181.498 | | x |
| 43 | 181.237 | | x |
| 44 | 180.935 | | x |

TABLE 3
Ragweed sensitized patients from USA (lane 45-58)
and Austria (lane 59-63): Blot III

| # | Patient | IgE-positive | IgE-negative |
|---|---|---|---|
| 45 | 2 | x | |
| 46 | 3 | x | |
| 47 | 5 | x | |
| 48 | 8 | x | |
| 49 | 14 | x | |
| 50 | 15 | x | |
| 51 | 17 | x | |
| 52 | 18 | x | |
| 53 | 19 | x | |
| 54 | 24 | x | |
| 55 | 25 | x | |
| 56 | 27 | x | |
| 57 | 28 | x | |
| 58 | 29 | x | |
| 59 | 1 | x | |
| 60 | 4 | x | |
| 61 | 7 | x | |
| 62 | 9 | x | |
| 63 | 10 | x | |

TABLE 4
Mugwort allergic patients from Salzburg (lane 64-68)
and Art v 1 T cell patients (lane 69-83): Blot IV

| # | Patient | IgE-positive | IgE-negative |
|---|---|---|---|
| 64 | 1 | x | |
| 65 | 2 | x | |
| 66 | 3 | x | |
| 67 | 4 | x | |
| 68 | 5 | x | |
| 69 | 1 | x | |
| 70 | 7 | x | |
| 71 | 10 | x | |
| 72 | 14 | x | |
| 73 | 15 | x | |
| 74 | 16 | x | |
| 75 | 22 | x | |
| 76 | 22 | x | |
| 77 | 23 | x | |
| 78 | 24 | x | |
| 79 | 25 | x | |
| 80 | 26 | x | |
| 81 | 27 | x | |
| 82 | 29 | x | |
| 83 | 31 | x | |

TABLE 5
Ragweed allergic patients form Canada (lane 84-107): Blot V

| # | Patient | IgE-positive | IgE-negative |
|---|---|---|---|
| 84 | C1-6 | x | |
| 85 | C1-8 | x | |
| 86 | C1-11 | | x |
| 87 | C1-12 | x | |
| 88 | C2-17 | x | |
| 89 | C2-18 | | x |
| 90 | C2-19 | | x |
| 91 | C3-25 | | x |
| 92 | C3-27 | | x |
| 93 | C4-33 | x | |
| 94 | C5-36 | | x |
| 95 | C5-38 | x | |
| 96 | C5-40 | | x |
| 97 | C5-43 | | x |
| 98 | C5-48 | | x |
| 99 | C5-52 | | x |
| 100 | C5-55 | | x |
| 101 | C5-56 | x | |
| 102 | C6-59 | | x |
| 103 | C6-61 | | x |

TABLE 6
Ragweed allergic patients from Canada
and Austria (lane 108-117): Blot VI

| # | Patient | IgE-positive | IgE-negative |
|---|---|---|---|
| 104 | C6-68 | | x |
| 105 | C6-69 | | x |
| 106 | C11-78 | | x |
| 107 | C11-82 | | x |
| 108 | 5 | | x |
| 109 | 10 | | x |
| 110 | 11 | | x |
| 111 | 12 | | x |
| 112 | 13 | | x |
| 113 | 14 | | x |
| 114 | 15 | | x |
| 115 | 16 | | x |
| 116 | 17 | | x |
| 117 | 18 | | x |

TABLE 7
Negative controls: Blot VII (2^{nd}antibody only!!)

| # | Patient | IgE-positive | IgE-negative |
|---|---|---|---|
| 118 | — | | x |
| 119 | — | | x |
| 120 | — | | x |
| 121 | — | | x |
| 122 | — | | x |
| 123 | — | | x |

Results of the Tested Patient Sera:

Mugwort allergic patients from Austria (total amount of tested patient sera=61)
IgE-positive: 36=59%
IgE-negative: 25=41%

Ragweed allergic patients from USA, Canada and Austria (total amount of tested patient sera=56)
IgE-positive: 27=48%
IgE-negative: 29=52%
   Overall Results:

Total amount of available sera from mugwort allergic patients: 73
IgE-positive: 36=49.4%
IgE-negative: 37=50.6%

Total amount of available sera from ragweed allergic patients: 130
IgE-positive: 27=20.8%
IgE-negative: 103=79.2%

Almost 50% of the mugwort sensitized patients and around 20% of the ragweed sensitized patients have IgE antibodies against the 40.9 kDa allergen. This is a relatively high percentage of patients sensitized to this mugwort pollen allergen. Consequently it is important to add this molecule to a vaccine preparation for treatment of these patients' allergy. Novel approaches for allergy therapy go in the direction of patient-tailored vaccine preparations.

1. A polypeptide selected from the group consisting of:
a) polypeptides comprising a fragment of at least 18 consecutive amino acids of the amino acid sequence as shown in SEQ ID NO:1;
b) polypeptides comprising an amino acid sequence which has an identity of at least 70% to the amino acid sequence as shown in SEQ ID NO:1; and
c) polypeptides consisting of at least 8 contiguous amino acids of the amino acid sequence as shown in SEQ ID NO:1.

2. A polypeptide according to claim 1, comprising a fragment of the amino acid sequence as shown in SEQ ID NO:1 wherein said fragment is capable of binding to IgE antibodies from an individual being allergic against mugwort pollen.

3. A polypeptide, comprising the amino acid sequence as shown in SEQ ID NO:1.

4. A polypeptide of claim 1, characterized in that it is capable of binding to IgE antibodies from an individual being allergic against ragweed pollen.

5. A polynucleotide selected from the group consisting of
a) polynucleotides encoding the amino acid sequence as shown in SEQ ID NO:1;
b) polynucleotides encoding a polypeptide as claimed in any one of claims 1, 2, 3, 4, 19, and 20; and
c) polynucleotides comprising a nucleotide sequence which has an identity of at least 75% to the nucleotide sequence as shown in SEQ ID NO:2;
or the complementary strand of such polynucleotide.

6. The polynucleotide of claim 5 comprising the nucleotide sequence as shown in SEQ ID NO:2 or the nucleotide sequence as shown in SEQ ID NO:3.

7. A plasmid or a vector comprising a polynucleotide of claim 5.

8. A cell containing a plasmid or a vector of claim 7 or 21.

9. A cell according to claim 8, which is selected from the group consisting of plant cells, bacterial cells and yeast cells.

10. A method for the preparation of a polypeptide comprising of culturing cells as claimed in claim 8, 9, 22, 23, 24, or 25 under conditions appropriate for the expression of the polypeptide and optionally subsequently recovering the polypeptide.

11. The method of claim 10, wherein the cells are opened and the polypeptide is recovered using affinity chromatography.

12. An antibody capable of binding to a polypeptide of any one of claims 1, 2, 3, 4, 19, and 20.

13. An antibody of claim 12, which is capable of binding to one or several of the polypeptides selected from the group consisting of Amb a I.1, Amb a I.2, Amb a I.3 and Amb a 2.

14. An antibody of claim 12, which does not bind to any one of the polypeptides selected from the group consisting of Amb a I.1, Amb a I.2, Amb a I.3 and Amb a 2.

15. A pharmaceutical composition comprising a polypeptide as claimed in any one of claims 1, 2, 3, 4, 19, and 20.

16. A method of treating, preventing, or diagnosing an allergic disorder by administering a medicament comprising a polypeptide as claimed in any one of claims 1, 2, 3, 4, 19, and 20.

17. The method of claim 16 wherein the medicament is administered to an individual to be desensitized.

18. A kit for the diagnosis, treatment, or prevention of an allergic disorder comprising a polypeptide as claimed in any one of claims 1, 2, 3, 4, 19, and 20.

19. The polypeptide of claim 2, wherein the polypeptide is capable of binding to IgE antibodies from an individual being allergic against ragweed pollen.

20. The polypeptide of claim 3, wherein the polypeptide is capable of binding to IgE antibodies from an individual being allergic against ragweed pollen.

21. A plasmid or a vector comprising a polynucleotide of claim 6.

22. A cell containing a-polynucleotide as claimed in claim 5.

23. A cell containing a polynucleotide as claimed in claim 6.

24. A cell according to claim 23, which is selected from the group consisting of plant cells, bacterial cells and yeast cells.

25. A cell according to claim 24, which is selected from the group consisting of plant cells, bacterial cells and yeast cells.

26. A pharmaceutical composition comprising a polynucleotide as claimed in claim 5.

27. A pharmaceutical composition comprising a polynucleotide as claimed in claim 6.

28. A pharmaceutical composition comprising an antibody as claimed in claim 12.

29. A pharmaceutical composition comprising an antibody as claimed in claims 13 or 14.

30. A method of treating, preventing, or diagnosing an allergic disorder comprising administering a polynucleotide of claim 5.

31. A method of treating, preventing, or diagnosing an allergic disorder comprising administering a polynucleotide of claim 6.

32. A method of treating, preventing, or diagnosing an allergic disorder comprising administering an antibody of claim 12.

33. A method of treating, preventing, or diagnosing an allergic disorder comprising administering an antibody of claim 13 or 14.

34. A kit for the diagnosis, treatment, or prevention of an allergic disorder comprising a polynucleotide as claimed in claim 5.

35. A kit for the diagnosis, treatment, or prevention of an allergic disorder comprising a polynucleotide as claimed in claim 6.

36. A kit for the diagnosis, treatment, or prevention of an allergic disorder comprising an antibody of claim 12.

37. A kit for the diagnosis, treatment, or prevention of an allergic disorder comprising an antibody of claim 13 or 14.
